(19) United States
(12) Patent Application Publication
(10) Pub. No.: US 2019/0117167 A1
(43) Pub. Date: Apr. 25, 2019

(54) IMAGE PROCESSING APPARATUS, LEARNING DEVICE, IMAGE PROCESSING METHOD, METHOD OF CREATING CLASSIFICATION CRITERION, LEARNING METHOD, AND COMPUTER READABLE RECORDING MEDIUM

(71) Applicant: OLYMPUS CORPORATION, Tokyo (JP)

(72) Inventors: Toshiya KAMIYAMA, Tokyo (JP); Yamato KANDA, Tokyo (JP)

(73) Assignee: OLYMPUS CORPORATION, Tokyo (JP)

(21) Appl. No.: 16/217,161

(22) Filed: Dec. 12, 2018

Related U.S. Application Data
(63) Continuation of application No. PCT/JP2016/068877, filed on Jun. 24, 2016.

(51) Int. Cl. A61B 5/00; A61B 1/267; A61B 1/273; A61B 1/307; G06K 9/62; G06K 9/66

(52) U.S. Cl. CPC A61B 5/7264; A61B 1/267; G06K 9/66; A61B 1/307; G06K 9/6267; A61B 1/2736

(57) ABSTRACT

An image processing apparatus includes: a memory; and a processor comprising hardware, the processor being configured to output a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group, wherein the similar image group is different from the image group to be classified in the main learning.

### CROSS-REFERENCE TO RELATED APPLICATION

This application is a continuation of International Application No. PCT/JP2016/068877, filed on Jun. 24, 2016, the entire contents of which are incorporated herein by reference.

### BACKGROUND

The present disclosure relates to an image processing apparatus, a learning device, an image processing method, a method of creating a classification criterion, a learning method, and a computer readable recording medium.

Recently, in a learning device that performs learning of a classifier using large volumes of data, in order to avoid overfitting in learning of a small number of data sets, a learning method is known where preliminary learning of a classifier is performed using a large number of general object image data sets such as ImageNet, followed by main learning using a small number of data sets (see ulkit Agrawal, et. al “Analyzing the Performance of Multilayer Neural Networks for Object Recognition”, arXiv: 1407.1610V2, arXiv. org, (22, Sep. 2014)).

### SUMMARY

An image processing apparatus according to one aspect of the present disclosure includes: a memory; and a processor comprising hardware, the processor being configured to output a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group, wherein the similar image group is different from the image group to be classified in the main learning.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating a configuration of a learning device according to a first embodiment;

FIG. 2 is a flowchart illustrating an outline of processing executed by the learning device according to the first embodiment;

FIG. 3 is a flowchart illustrating an outline of preliminary learning processing in FIG. 2;

FIG. 4 is a flowchart illustrating an outline of preliminary learning medical image acquiring processing in FIG. 3;

FIG. 5 is a flowchart illustrating an outline of main learning in FIG. 2;

FIG. 6 is a flowchart illustrating an outline of preliminary learning medical image acquiring processing according to a first modification of the first embodiment;

FIG. 7 is a flowchart illustrating an outline of preliminary learning processing executed by a preliminary learning unit according to a second modification of the first embodiment;

FIG. 8 is a flowchart illustrating an outline of medical image acquiring processing in FIG. 7;

FIG. 9 is a flowchart illustrating an outline of preliminary learning processing executed by a preliminary learning unit according to a third modification of the first embodiment;

FIG. 10 is a flowchart illustrating an outline of medical image acquiring processing in FIG. 9;

FIG. 11 is a block diagram illustrating a configuration of a learning device according to a second embodiment;

FIG. 12 is a flowchart illustrating an outline of processing executed by the learning device according to the second embodiment;

FIG. 13 is a flowchart illustrating an outline of basic learning processing in FIG. 12;

FIG. 14 is a block diagram illustrating a configuration of an image processing apparatus according to a third embodiment; and

FIG. 15 is a flowchart illustrating an outline of processing executed by the image processing apparatus according to the third embodiment.

### DETAILED DESCRIPTION

An image processing apparatus, a learning method, and a program including a learning device according to embodiments will be described below with reference to the drawings. The present disclosure is not limited by these embodiments. In addition, identical sections in descriptions of the drawings are denoted by identical reference numerals.

### First Embodiment

Configuration of Learning Device

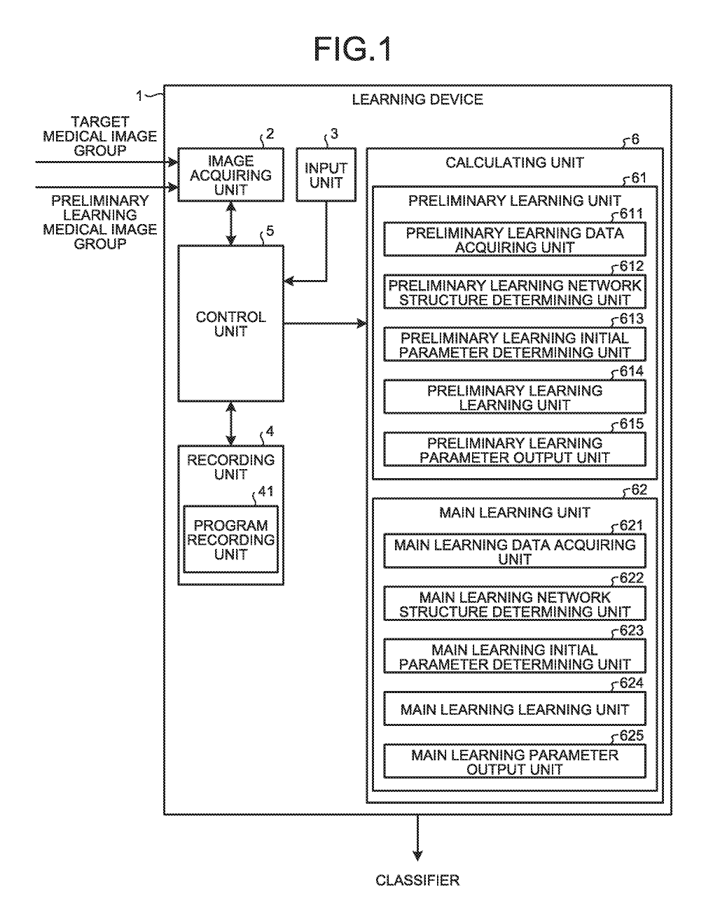

FIG. 1 is a block diagram illustrating a configuration of a learning device according to a first embodiment. A learning device 1 according to the first embodiment performs, for example, preliminary learning based on a similar image group similar in at least one of characteristics of a shape of an object in a target medical image group to be learned that is obtained by capturing a lumen in a living body with an endoscope (an endoscope scope such as a flexible endoscope or a rigid endoscope) or a capsule endoscope (hereinafter collectively referred to as merely “endoscope”), a tissue structure of the object, and an imaging system of the endoscope, followed by main learning based on the target medical image group to be learned. Here, a medical image is usually a color image having pixel levels (pixel values) for wavelength components of R (red), G (green), and B (blue) at each pixel position.

The learning device 1 illustrated in FIG. 1 includes an image acquiring unit 2 that acquires, from an endoscope or from outside, target medical image group data corresponding to a medical image group captured with an endoscope and preliminary learning medical image group data, an input unit 3 that receives an input signal input by an external operation, a recording unit 4 that records image data acquired by the image acquiring unit 2 and various programs, a control unit 5 that controls operation of the learning device 1 as a whole, and a calculating unit 6 that performs learning based on target medical image group data and preliminary learning medical image group data acquired by the image acquiring unit 2.

The image acquiring unit 2 is appropriately configured according to an aspect of a system including an endoscope. For example, when a portable recording medium is used for delivering image data to and from an endoscope, the image acquiring unit 2 is configured to have this recording medium detachably mounted and serve as a reader that reads recorded image data. Further, when acquiring image data captured with an endoscope via a server, the image acquiring unit 2 includes a communication device or the like bidirectionally communicable with this server and acquires image data through data communication with the server. Furthermore, the image acquiring unit 2 may include an interface device or the like through which image data are input from a recording device that records image data captured with an endoscope via a cable.

The input unit 3 is realized by, for example, input devices such as a keyboard, a mouse, a touch panel, and various switches and outputs an input signal received according to an external operation to the control unit 5.

The recording unit 4 is realized by various IC memories such as a flash memory, a read only memory (ROM), and a random access memory (RAM) and a hard disk or the like incorporated or connected by data communication terminals. In addition to image data acquired by the image acquiring unit 2, the recording unit 4 records a program for causing the learning device 1 to operate as well as to execute various functions, data used during execution of this program, and the like. For example, the recording unit 4 records a program recording unit 41 for performing main learning using a target medical image group after preliminary learning is performed using a preliminary learning medical image group, information on a network structure in order for the calculating unit 6 described later to perform learning, or the like.

The control unit 5 is realized by using a central processing unit (CPU) or the like and by reading various programs recorded in the recording unit 4, provides instructions, transfers data, or the like to each unit that constitutes the learning device 1 according to image data input from the image acquiring unit 2, an input signal input from the input unit 3, or the like to totally control operation of the learning device 1 as a whole.

The calculating unit 6 is realized by a CPU or the like and executes learning processing by reading a program from the program recording unit 41 recorded by the recording unit 4.

Configuration of Calculating Unit

Next, a detailed configuration of the calculating unit 6 will be described. The calculating unit 6 includes a preliminary learning unit 61 that performs preliminary learning based on a preliminary learning medical image group and a main learning unit 62 that performs main learning based on a target medical image group.

The preliminary learning unit 61 includes a preliminary learning data acquiring unit 611 that acquires preliminary learning data, a preliminary learning network structure determining unit 612 that determines a network structure for preliminary learning, a preliminary learning initial parameter determining unit 613 that determines an initial parameter of a network for preliminary learning, a preliminary learning learning unit 614 that performs preliminary learning, and a preliminary learning parameter output unit 615 that outputs a parameter learned through preliminary learning.

The main learning unit 62 includes a main learning data acquiring unit 621 that acquires main learning data, a main learning network structure determining unit 622 that determines a network structure for main learning, a main learning initial parameter determining unit 623 that determines an initial parameter of a network for main learning, a main learning learning unit 624 that performs main learning, and a main learning parameter output unit 625 that outputs a parameter learned through main learning.

Processing by Learning Device

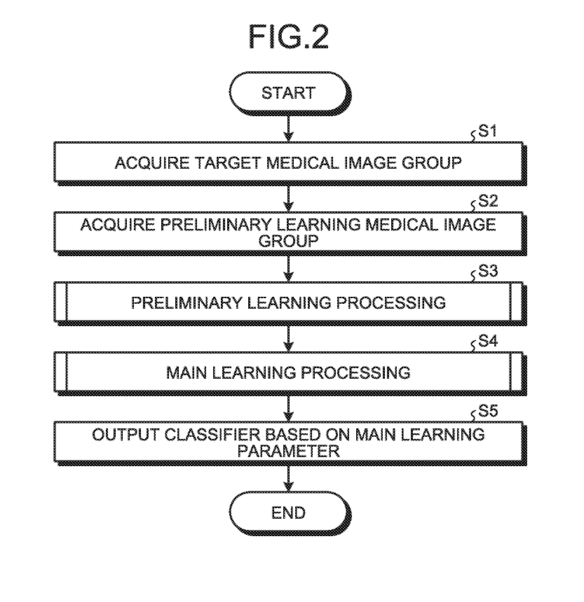

Next, processing executed by the learning device 1 will be described. FIG. 2 is a flowchart illustrating an outline of the processing executed by the learning device 1.

As illustrated in FIG. 2, first, the image acquiring unit 2 acquires a target medical image group to be processed (Step S1) and acquires a preliminary learning medical image group to be processed during preliminary learning (Step S2).

Subsequently, the preliminary learning unit 61 executes preliminary learning processing for performing preliminary learning based on the preliminary learning medical image group acquired by the image acquiring unit 2 (Step S3).

Preliminary Learning Processing

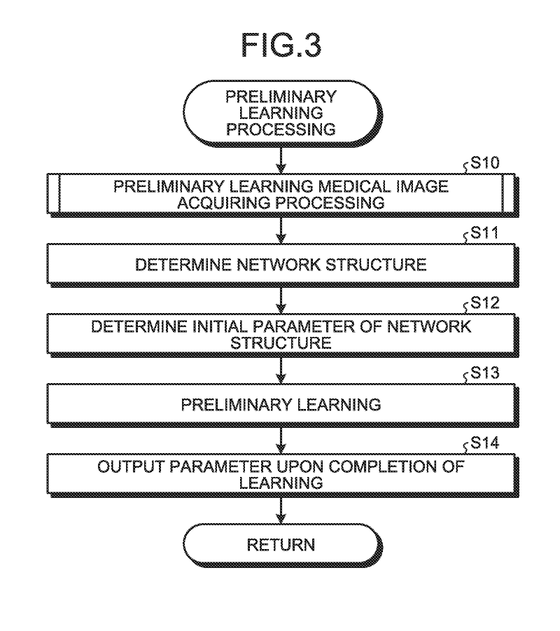

FIG. 3 is a flowchart illustrating an outline of the preliminary learning processing in Step S3 in FIG. 2.

As illustrated in FIG. 3, the preliminary learning data acquiring unit 611 executes preliminary learning medical image acquiring processing for acquiring a preliminary learning medical image group recorded in the recording unit 4 (Step S10). Here, a preliminary learning medical image group is a medical image group different from a target medical image group in main learning and similar to characteristics of the medical image group. Specifically, a preliminary learning medical image group is a medical image group similar in a shape of an object. For example, shapes of an object include a tubular structure. A tubular structure unique in a human body in a medical image generates a special circumstance when capturing a way for a light source to spread by an endoscope, a way for shadows to occur, distortions of an object due to depth, or the like. To preliminarily learn this special circumstance, a general object image group is considered insufficient. Thus, in the first embodiment, by learning a medical image group similar to the special circumstance described above in preliminary learning, it is possible to acquire a parameter tailored to the special circumstance in preliminary learning. As a result, preliminary learning may be performed with high accuracy. Specifically, in the first embodiment, a group of images of another organ in a lumen in a living body is used as a preliminary learning medical image group. For example, in the first embodiment, when a target medical image group is a medical image group of small intestine captured with a small intestine endoscope (hereinafter referred to as “small intestine endoscopic image group”), a medical image group of large intestine captured with a large intestine endoscope (hereinafter referred to as “large intestine endoscopic image group”) that is generally considered to have a larger number of inspections (number of cases) is set as a preliminary learning medical image group.

Preliminary Learning Medical Image Acquiring Processing

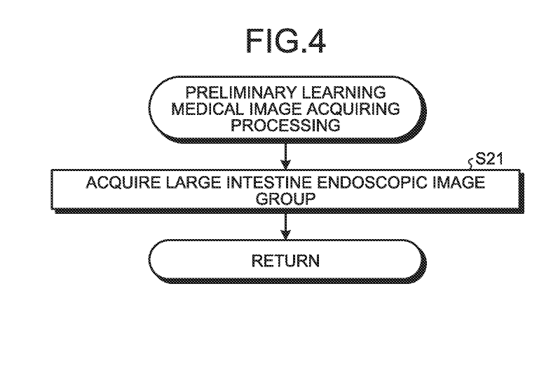

FIG. 4 is a flowchart illustrating an outline of the preliminary learning medical image acquiring processing in Step S10 in FIG. 3.

As illustrated in FIG. 4, when a target medical image group corresponding to an instruction signal input from the input unit 3 is a small intestine endoscopic image group, the preliminary learning data acquiring unit 611 acquires a large intestine endoscopic image group from the recording unit 4 as a preliminary learning medical image group (Step S21). In this case, the preliminary learning data acquiring unit 611 acquires the large intestine endoscopic image group divided into arbitrary classes. For example, the preliminary learning data acquiring unit 611 acquires a small intestine endoscopic image group in main learning divided into two classes, normal or abnormal, in order to detect abnormality. Therefore, the preliminary learning data acquiring unit 611 similarly acquires the large intestine endoscopic image group as a preliminary learning medical image group, divided into two classes, normal or abnormal. Thus, due to commonness in structure peculiar to inside of a human body, a lumen, even when the number of the target medical image groups is small, the preliminary learning data acquiring unit 611 may effectively learn the special circumstance described above in preliminary learning. After Step S21, the learning device 1 returns to the preliminary learning processing in FIG. 3.

Returning to FIG. 3, descriptions from Step S11 will be continued.

In Step S11, the preliminary learning network structure determining unit 612 determines a structure of a network used for preliminary learning. For example, the preliminary learning network structure determining unit 612 determines a convolutional neural network (CNN) that is a type of a neural network (NN) as a structure of a network used for preliminary learning (reference: Springer Japan, “Pattern Recognition and Machine Learning”, p. 270-272 (Chapter 5 Neural Network 5.5.6 Convolution neural network)). Here, as a structure of the CNN, the preliminary learning network structure determining unit 612 may appropriately select a structure for ImageNet installed in a tutorial of image recognition root Caffe of deep learning (reference: http://caffe.berkeleyvision.org/), a structure for CIFAR-10, or the like.

Subsequently, the preliminary learning initial parameter determining unit 613 determines an initial parameter of the network structure determined by the preliminary learning network structure determining unit 612 (Step S12). In the first embodiment, the preliminary learning initial parameter determining unit 613 determines a random value as an initial parameter.

Thereafter, the preliminary learning learning unit 614 inputs the preliminary learning medical image acquired by the preliminary learning data acquiring unit 611 and performs preliminary learning based on the network structure determined by the preliminary learning network structure determining unit 612 using the initial value determined by the preliminary learning initial parameter determining unit 613 (Step S13).

Here, details of preliminary learning by the preliminary learning learning unit 614 will be described. Hereinafter, a case where the preliminary learning network structure determining unit 612 determines the CNN as a network structure will be described (reference: A Concept of Deep Learning viewed from Optimization).

The CNN is a type of model and represents a prediction function by synthesis of a multiple of nonlinear transformations. The CNN is defined for input x=h₀ with f₁, . . . , f_{L} as a nonlinear function as in Formula 1 below.

hᵢ=fᵢ(zᵢ), zᵢ=Wᵢhᵢ₋₁+bᵢ(i=l, . . . , L) (1)

Wᵢ is a connection weighting matrix, and bᵢ is a bias vector, both of which are parameters to be learned. In addition, components of each hᵢ are called units. Each nonlinear function fᵢ is an activating function and has no parameter. A loss function is defined for output h_{L} of the NN. In the first embodiment, a cross entropy error is used. Specifically, Formula 2 below is used.

l(h_{L})=Σᵢ(yᵢlog h_{L, i}+(1−yᵢ)log(1−h_{L, i})) (2)

In this case, since h_{L} needs to be a probability vector, a softmax function is used as an activating function of a final layer. Specifically, Formula 3 below is used.

f(x₁)=(exp(xᵢ)/Σⱼexp(xⱼ))ᵢ₌₁^{d}(i=1, . . . , d) (3)

Here, the activating function is the number of units of an output layer. This is an example of an activating function that is unable to be decomposed into real-valued functions for each unit. A method of optimizing the NN is mainly a method based on gradient. A gradient of transmission l=l(h_{L}) for certain data may be calculated by applying a chain rule to Formula 1 described above as follows.

∇_{z}_{ᵢ}l=f′₁(zᵢ)∇_{z}_{ᵢ}l, ∇ₕ_{ᵢ₌ₗ}=Wᵢ^{T∇}_{z}_{ᵢ}l (4)

∇_{W}_{ᵢ}l=∇_{z}_{ᵢ}lhᵢ₋₁^{T}, ∇_{b}_{ᵢ}l=∇_{z}_{ᵢ}l (5)

With ∇_{HL}l as a starting point, ∇_{HL}l is calculated in the order of i=L−1, . . . , 2 using Formula 4 described above, and a gradient of a parameter is derived for each layer using Formula 5. This algorithm is called an error back propagation algorithm. Using this error back propagation algorithm, learning is pursued so as to minimize a loss function. In the first embodiment, a function max (0, x) is used as an activating function. This function is called a rectified linear unit (ReLU), a rectifier, or the like. Despite a disadvantage that a range is not bounded, the ReLU is advantageous in optimization because a gradient propagates without attenuation for a unit taking a positive value (reference: Springer Japan, “Pattern Recognition and Machine Learning” p. 242-250 (Chapter 5 Neural Network 5.3. Error back propagation)). The preliminary learning learning unit 614 sets a learning completion condition to, for example, the number of learning times and completes preliminary learning when the set number of learning times is reached.

After Step S13, the preliminary learning parameter output unit 615 outputs a parameter upon completion of the preliminary learning performed by the preliminary learning learning unit 614 (Step S14). After Step S14, the learning device 1 returns to FIG. 2.

Returning to FIG. 2, descriptions from Step S4 will be continued.

In Step S4, the main learning unit 62 executes main learning processing for performing main learning based on the target medical image group acquired by the image acquiring unit 2.

Main Learning Processing

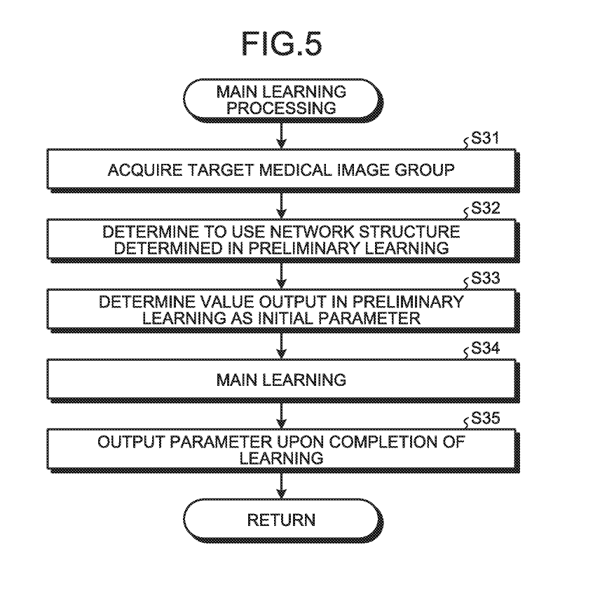

FIG. 5 is a flowchart illustrating an outline of the main learning in Step S4 in FIG. 2.

As illustrated in FIG. 5, the main learning data acquiring unit 621 acquires a target medical image group recorded in the recording unit 4 (Step S31).

Subsequently, the main learning network structure determining unit 622 determines the network structure determined by the preliminary learning network structure determining unit 612 in Step S11 described above as a network structure used in main learning (Step S32).

Thereafter, the main learning initial parameter determining unit 623 determines the value (parameter) output by the preliminary learning parameter output unit 615 in Step S14 described above as an initial parameter (Step S33).

Subsequently, the main learning learning unit 624 inputs the target medical image group acquired by the main learning data acquiring unit 621 and performs main learning based on the network structure determined by the main learning network structure determining unit 622 using the initial value determined by the main learning initial parameter determining unit 623 (Step S34).

Thereafter, the main learning parameter output unit 625 outputs a parameter upon completion of the main learning performed by the main learning learning unit 624 (Step S35). After Step S35, the learning device 1 returns to a main routine in FIG. 2.

Returning to FIG. 2, descriptions from Step S5 will be continued.

In Step S5, the calculating unit 6 outputs a classifier based on the parameter of the main learning toward outside.

According to the first embodiment described above, through preliminary learning by the preliminary learning unit 61 of a medical image different from a target medical image but similar in characteristics that a shape of an object in the target medical image is a tubular structure, followed by main learning of the target medical image by the main learning unit 62 with a preliminary learning result by the preliminary learning unit 61 as an initial value, a parameter for capturing image features of a luminal structure in a human body such as a way for a light source to spread, a way for shadows to occur, and distortions of an object due to depth is preliminarily learned. This allows for highly accurate learning. As a result, even with a small number of data sets, a classifier with high classification accuracy may be obtained.

First Modification of First Embodiment

Next, a first modification of the first embodiment will be described. The first modification of the first embodiment is different in the preliminary learning medical image acquiring processing executed by the preliminary learning data acquiring unit 611 according to the first embodiment described above. Hereinafter, only preliminary learning medical image acquiring processing executed by the preliminary learning data acquiring unit 611 according to the first modification of the first embodiment will be described. Configurations identical to those of the learning device 1 according to the first embodiment are denoted by identical reference numerals, and descriptions thereof will be omitted.

Preliminary Learning Medical Image Acquiring Processing

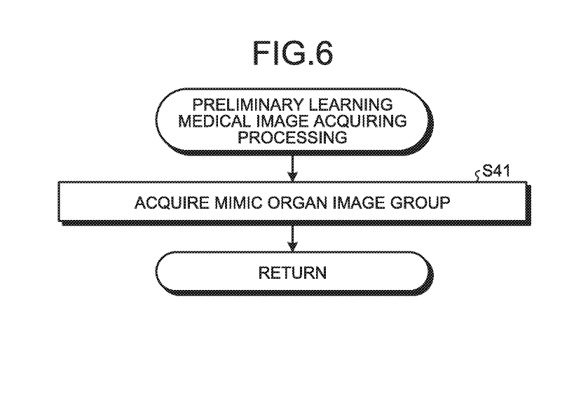

FIG. 6 is a flowchart illustrating an outline of the preliminary learning medical image acquiring processing according to the first modification of the first embodiment.

As illustrated in FIG. 6, when a target medical image group corresponding to an instruction signal input from the input unit 3 is a small intestine endoscopic image group, the preliminary learning data acquiring unit 611 acquires a mimic organ image group obtained by capturing a mimic organ that mimics a state of small intestine from the recording unit 4 as a preliminary learning medical image group (Step S41). Here, a mimic organ image group is so-called an image group obtained by capturing a living body phantom that mimics a state of small intestine with an endoscope or the like. In this case, the preliminary learning data acquiring unit 611 acquires a mimic organ image group divided into arbitrary classes. For example, usually, a small intestine endoscopic image group in main learning is divided into two classes, normal or abnormal, in order to detect abnormality. Therefore, the preliminary learning data acquiring unit 611 similarly acquires a mimic organ image group as a preliminary learning medical image group divided into two classes, normal or abnormal, by preparing a mucosal damaged condition in a living body phantom and capturing normal sites and mucosa damaged sites with an endoscope or the like. After Step S41, the learning device 1 returns to the preliminary learning processing in FIG. 3.

According to the first modification of the first embodiment described above, compared to an endoscopic image group of small intestine of which data are difficult to collect, a living body phantom may be captured any number of times, and thus, a structure peculiar to inside of a human body may be learned. Therefore, preliminary learning may be learned with high accuracy.

Second Modification of First Embodiment

Next, a second modification of the first embodiment will be described. The second modification of the first embodiment is different in the preliminary learning processing executed by the preliminary learning unit 61 according to the first embodiment described above. Hereinafter, preliminary learning processing executed by a preliminary learning unit according to the second modification of the first embodiment will be described. Configurations identical to those of the learning device 1 according to the first embodiment are denoted by identical reference numerals, and descriptions thereof will be omitted.

Preliminary Learning Processing

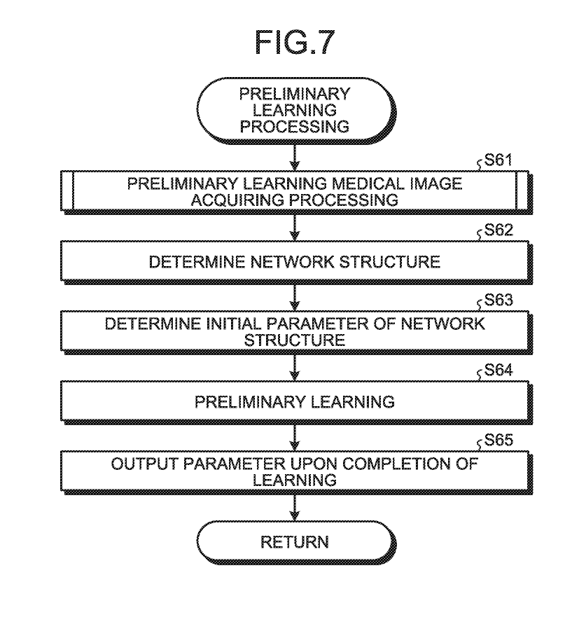

FIG. 7 is a flowchart illustrating an outline of the preliminary learning processing executed by the preliminary learning unit 61 according to the second modification of the first embodiment.

As illustrated in FIG. 7, first, the preliminary learning data acquiring unit 611 executes preliminary learning medical image acquiring processing for acquiring a preliminary learning medical image group recorded in the recording unit 4 (Step S61). Here, a preliminary learning medical image is a medical image different from a target medical image in main learning and similar to characteristics of the medical image. Specifically, a preliminary learning medical image is a medical image similar in tissue structure of an object in a target medical image in main learning. As the tissue structure of an object, for example, an organ system is identical. A tissue structure peculiar to inside of a human body generates many special circumstances when capturing with an endoscope or the like, such as an appearance of reflected light caused by a texture pattern and a fine structure. Thus, in the second modification of the first embodiment, by learning an image data group similar to the special circumstances described above in preliminary learning, it is possible to acquire a parameter tailored to the special circumstances in preliminary learning. As a result, preliminary learning may be performed with high accuracy. Specifically, in the second modification of the first embodiment, it is assumed that the organ system is common in any one of digestive, respiratory, urinary, and circulatory organs. When a target medical image is a small intestine endoscopic image, the preliminary learning data acquiring unit 611 acquires an image of a stomach that is also a digestive organ as a preliminary learning medical image used for preliminary learning.

Medical Image Acquiring Processing

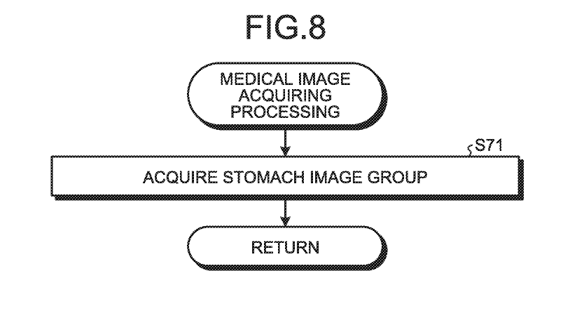

FIG. 8 is a flowchart illustrating an outline of the preliminary learning medical image acquiring processing described in Step S61 of FIG. 7.

As illustrated in FIG. 8, when a target medical image group corresponding to an instruction signal input from the input unit 3 is a small intestine endoscopic image group, the preliminary learning data acquiring unit 611 acquires a stomach image group that has a characteristic of being an identical digestive organ and that is different from an organ in the target medical image group from the recording unit 4 as a preliminary learning medical image group (Step S71). In this case, the preliminary learning data acquiring unit 611 arbitrarily sets the number of classes. After Step S71, the learning device 1 returns to FIG. 7. Steps S62 to S65 correspond to Steps S11 to S14 in FIG. 3 described above, respectively. After Step S65, the learning device 1 returns to the main routine of FIG. 2.

According to the second modification of the first embodiment described above, a mucosal structure peculiar to inside of a human body similar to features of the target medical image group is learned because of being an identical digestive organ. Therefore, through preliminary learning of a particularly controversial fine texture feature data in medical images, followed by main learning with a result of the preliminary learning as an initial value, it is possible to capture features of an image such as an appearance of reflected light caused by a texture pattern and a fine structure of a tissue structure in a human body, so that highly accurate learning may be performed.

Third Modification of First Embodiment

Next, a third modification of the first embodiment will be described. The third modification of the first embodiment is different in the preliminary learning processing executed by the preliminary learning unit 61 according to the first embodiment described above. Hereinafter, preliminary learning processing executed by preliminary learning processing unit according to the third modification of the first embodiment will be described. Configurations identical to those of the learning device 1 according to the first embodiment are denoted by identical reference numerals, and descriptions thereof will be omitted.

Preliminary Learning Processing

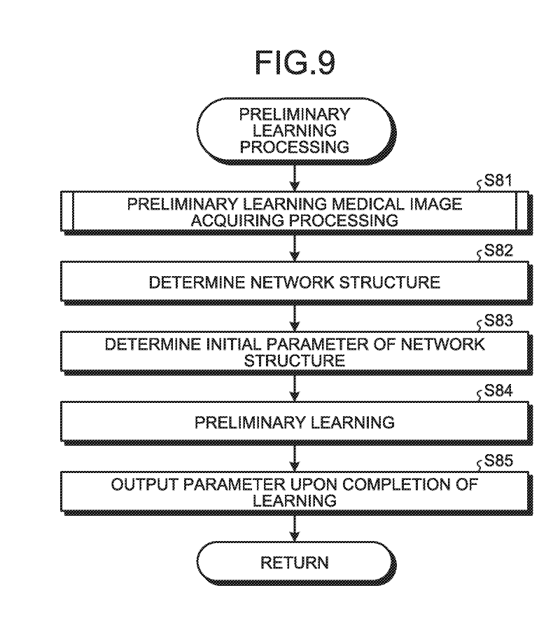

FIG. 9 is a flowchart illustrating an outline of the preliminary learning processing executed by the preliminary learning unit 61 according to the third modification of the first embodiment.

As illustrated in FIG. 9, first, the preliminary learning data acquiring unit 611 executes medical image acquiring processing for acquiring a medical image group for preliminary learning recorded in the recording unit 4 (Step S81). Here, a medical image group for preliminary learning is a medical image group different from a target medical image group in main learning and similar to characteristics of the medical image group. Specifically, a medical image group for preliminary learning is a medical image group similar in each of an imaging system (including an optical system and an illumination system) that captures a target medical image group in main learning and an object. Imaging systems include an imaging system of an endoscope. An endoscope that enters inside of a subject under study generates many special circumstances when capturing with an endoscope or the like, such as wide-angle inherent distortions in capturing, characteristics of an image sensor itself, and illumination characteristics due to illumination light. Thus, in the third modification of the first embodiment, through learning of an image group similar to the special circumstances described above in preliminary learning, it is possible to acquire a parameter tailored to the special circumstances in preliminary learning. As a result, preliminary learning may be performed with high accuracy. Specifically, in the third modification of the first embodiment, a medical image group that has an identical imaging system and that is obtained by capturing a mimic organ by this identical imaging system is used in preliminary learning. For example, when a target medical image group is an image group obtained by capturing a stomach with an endoscope for stomachs, the preliminary learning data acquiring unit 611 acquires an image group obtained by capturing a living body phantom that mimics a stomach with an endoscope for stomachs as a preliminary learning medical image group.

Medical Image Acquiring Processing

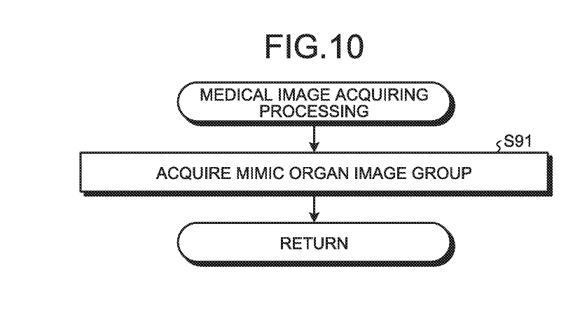

FIG. 10 is a flowchart illustrating an outline of the medical image acquiring processing described in Step S81 of FIG. 9.

As illustrated in FIG. 10, when a target medical image group corresponding to an instruction signal input from the input unit 3 is a stomach endoscopic image group captured with an endoscope for stomachs, the preliminary learning data acquiring unit 611 acquires a mimic organ image group with characteristics of having an identical imaging system as well as with characteristics identical to those of an organ of the target medical image from the recording unit 4 as a preliminary learning medical image group (Step S91). In this case, the number of classes is arbitrary for the mimic organ image group acquired by the preliminary learning data acquiring unit 611. The stomach endoscopic image group in main learning is categorized into two classes, normal or abnormal, in order to detect an abnormality. Therefore, it is preferred that the mimic organ image group in preliminary learning similarly be categorized into two classes, by preparing a mucosal damaged condition in a living body phantom and regarding the mucosal damaged condition captured as abnormal and others captured as normal. As a result, compared to an endoscopic image group of an actual stomach of which data are difficult to collect, a living body phantom may be captured any number of times and thus, may be learned by an identical imaging system while corresponding to a small amount of data. Therefore, preliminary learning may be performed with high accuracy. After Step S91, the learning device 1 returns to FIG. 9. Steps S82 to S85 correspond to Steps S11 to S14 in FIG. 3 described above, respectively. After Step S85, the learning device 1 returns to the main routine in FIG. 2.

According to the third modification of the first embodiment described above, through preliminary learning by the preliminary learning unit 61 of a medical image group different from the target medical image group and similar to characteristics of the target medical image group, followed by main learning of the target medical image group by the main learning unit 62 with a preliminary learning result by the preliminary learning unit 61 as an initial value, it is possible to preliminarily learn a parameter for capturing image features of an endoscope that captures inside of a human body, such as wide-angle inherent distortions in capturing, characteristics of an imaging sensor itself, and illumination characteristics due to illumination light. This allows for highly accurate learning.

### Second Embodiment

Next, a second embodiment will be described. An image processing apparatus according to the second embodiment is different in configuration from the learning device 1 according to the first embodiment described above. Specifically, in the first embodiment, main learning is performed after preliminary learning. However, in the second embodiment, basic learning is further performed before preliminary learning. Hereinafter, a configuration of the image processing apparatus according to the second embodiment will be described, followed by description of processing executed by a learning device according to the second embodiment. Configurations identical to those of the learning device 1 according to the first embodiment are denoted by identical reference numerals, and descriptions thereof will be omitted.

Configuration of Image Processing Apparatus

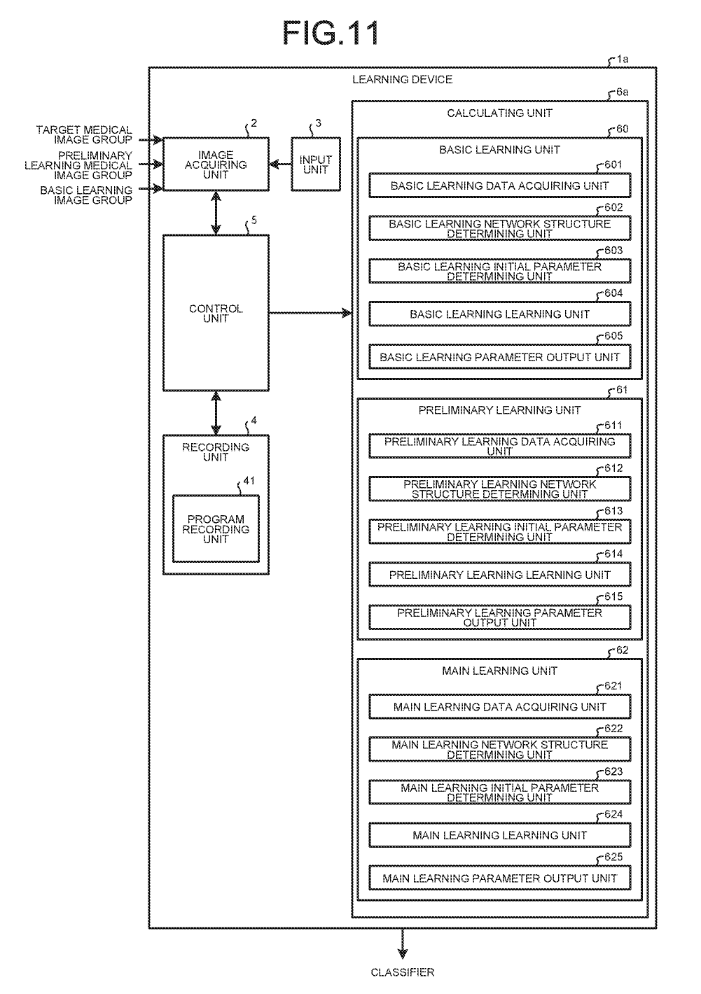

FIG. 11 is a block diagram illustrating a configuration of a learning device according to the second embodiment. A learning device la illustrated in FIG. 11 includes a calculating unit 6a in place of the calculating unit 6 of the learning device 1 according to the first embodiment.

### Configuration of Calculating Unit

In addition to the configuration of the calculating unit 6 according to the first embodiment, the calculating unit 6a further includes a basic learning unit 60.

The basic learning unit 60 performs basic learning. Here, basic learning is to learn using general large-scale data (general large-scale image group) different from a target medical image group before preliminary learning. General large-scale data include ImageNet. Through CNN learning with a general large-scale image group, part of the network mimics initial visual cortex of mammals (reference: Deep Learning and Image Recognition; Foundation and Recent Trends, Takayuki Okaya). In the second embodiment, preliminary learning is executed with an initial value that mimics the initial visual cortex described above. This may improve accuracy compared with a random value.

The basic learning unit 60 includes a basic learning data acquiring unit 601 that acquires a basic learning image group, a basic learning network structure determining unit 602 that determines a network structure for basic learning, a basic learning initial parameter determining unit 603 that determines an initial parameter of a basic learning network, a basic learning learning unit 604 that performs basic learning, and a basic learning parameter output unit 605 that outputs a parameter learned through basic learning.

Processing by Learning Device

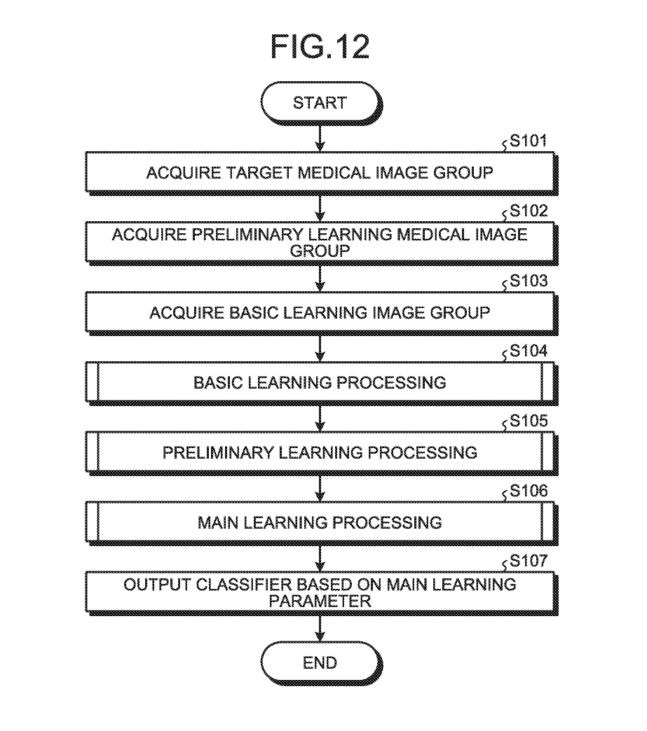

Next, processing executed by the learning device la will be described. FIG. 12 is a flowchart illustrating an outline of the processing executed by the learning device 1a. In FIG. 12, Steps 5101 and 5102 and Steps 5105 to 5107 correspond to Steps S1 to S5 in FIG. 2 described above, respectively.

In Step S103, the image acquiring unit 2 acquires a basic learning image group for performing basic learning.

Subsequently, the basic learning unit 60 executes basic learning processing for performing basic learning (Step S104).

Basic Learning Processing

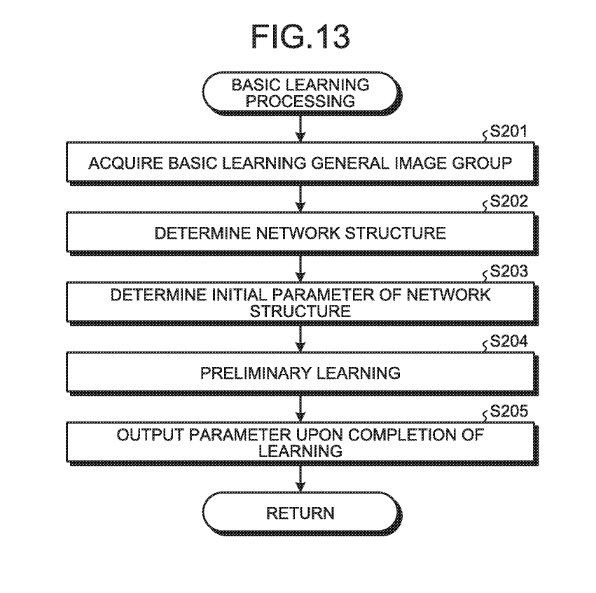

FIG. 13 is a flowchart illustrating an outline of the basic learning processing in Step S104 in FIG. 12 described above.

As illustrated in FIG. 13, the basic learning data acquiring unit 601 acquires a basic learning general image group recorded in the recording unit 4 (Step S201).

Subsequently, the basic learning network structure determining unit 602 determines a network structure used for learning (Step S202). For example, the basic learning network structure determining unit 602 determines a CNN as a network structure used for learning.

Thereafter, the basic learning initial parameter determining unit 603 determines an initial parameter of the network structure determined by the basic learning network structure determining unit 602 (Step S203). In this case, the basic learning initial parameter determining unit 603 determines a random value as an initial parameter.

Subsequently, the basic learning unit 604 inputs a general image group for the basic learning acquired by the basic learning data acquiring unit 601 and performs preliminary learning using the initial value determined by the basic learning initial parameter determining unit 603 based on the network structure determined by the basic learning network structure determining unit 602 (Step S204).

Thereafter, the basic learning parameter output unit 605 outputs a parameter upon completion of basic learning performed by the basic learning learning unit 604 (Step S205). After Step S205, the learning device la returns to a main routine of FIG. 12.

According to the second embodiment described above, through basic learning by the basic learning unit 60 of a large number of general images different from a target medical image before preliminary learning, it is possible to obtain an initial value effective during preliminary learning. This allows for highly accurate learning.

Third Embodiment

Next, a third embodiment will be described. An image processing apparatus according to the third embodiment is different in configuration from the learning device 1 according to the first embodiment described above. Specifically, in the first embodiment, a learning result is output to a classifier, but in the third embodiment, a classifier is provided in the image processing apparatus and classifies a classification target image based on a main learning output parameter. Hereinafter, a configuration of the image processing apparatus according to the third embodiment will be described, followed by description of processing executed by the image processing apparatus according to the third embodiment.

Configuration of Image Processing Apparatus

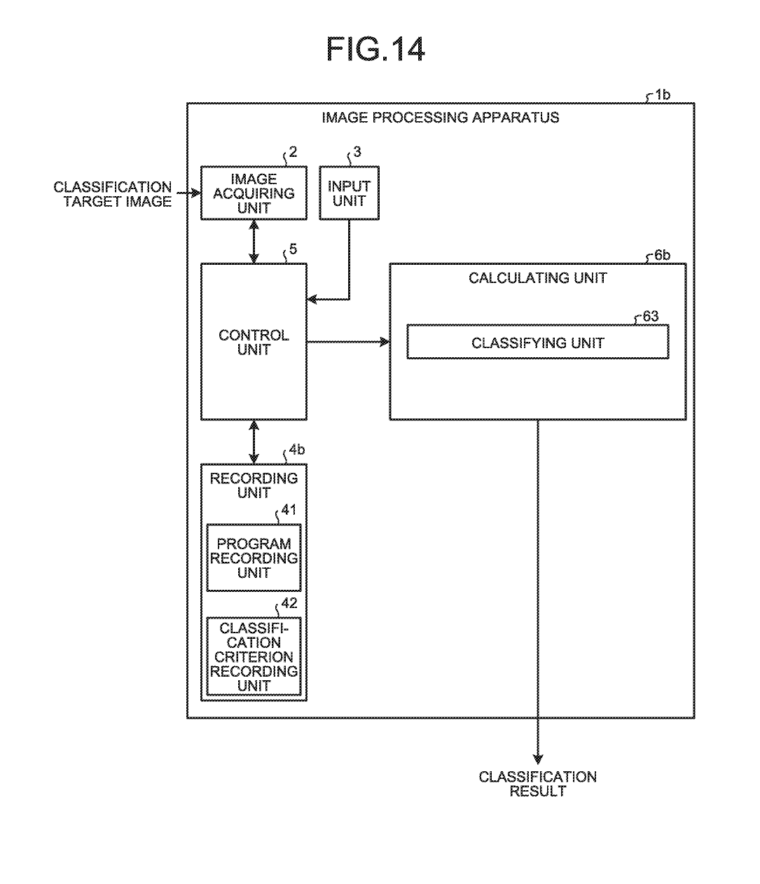

FIG. 14 is a block diagram illustrating the configuration of the image processing apparatus according to the third embodiment. An image processing apparatus 1b illustrated in FIG. 14 includes a calculating unit 6b and a recording unit 4b in place of the calculating unit 6 and the recording unit 4 of the learning device 1 according to the first embodiment.

In addition to the configuration of the recording unit 4 according to the first embodiment, the recording unit 4b has a classification criterion recording unit 42 that records a main learning output parameter (main learning result) that is a classification criterion created by the learning devices 1 and 1a of the first and the second embodiments described above.

Configuration of Calculating Unit

The calculating unit 6b has a classifying unit 63. The classifying unit 63 outputs a result of classifying a classification target image group based on the main learning output parameter that is a classification criterion recorded by the classification criterion recording unit 42.

Processing by Image Processing Apparatus

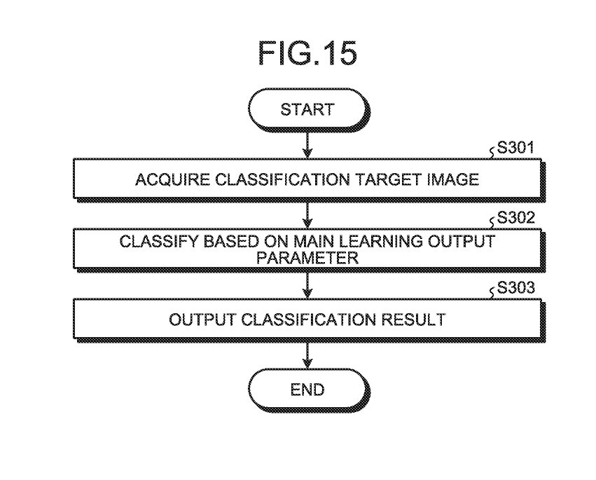

FIG. 15 is a flowchart illustrating an outline of processing executed by the image processing apparatus 1b. As illustrated in FIG. 15, the image acquiring unit 2 acquires a classification target image (Step S301).

Subsequently, the classifying unit 63 classifies a classification target image based on the main learning output parameter that is a classification criterion recorded by the classification criterion recording unit 42 (Step S302). Specifically, when carrying out two-class categorization in main learning such as whether a small intestine endoscopic image is normal or abnormal, the classifying unit 63 creates a classification criterion based on a network with a parameter learned in main learning set as an initial value and carries out, based on this created classification criterion, two-class categorization whether a new classification target image is normal or abnormal.

Thereafter, the calculating unit 6b outputs a classification result based on the categorization result by the classifying unit 63 (Step S303). After Step S303, the present processing is completed.

According to the third embodiment described above, the classifying unit 63 classifies a new classification target image using a network with a parameter learned in main learning set as an initial value. Therefore, a result of learning with high accuracy may be applied to a classification target image.

### Other Embodiments

In the present disclosure, an image processing program recorded in a recording device may be realized by being executed on a computer system such as a personal computer or a workstation. Further, such a computer system may be used by being connected to a device such as other computer systems or servers via a public line such as a local area network (LAN), a wide area network (WAN), or the Internet. In this case, the learning devices and the image processing apparatuses according to the first and the second embodiments and their modifications may acquire data of intraluminal images through these networks, output image processing results to various output devices such as a viewer and a printer connected through these networks, or store image processing results on a storage device connected through these networks, for example, a recording medium readable by a reader connected to a network.

In the descriptions of the flowcharts in the present specification, context of processings between the steps is clearly indicated by using expressions such as “first”, “thereafter”, and “subsequently”, but processing sequences necessary to implement the present disclosure are not uniquely determined by those expressions. In other words, processing sequences in the flowcharts described in the present specification may be changed within a range without inconsistency.

The present disclosure is not limited to the first to the third embodiments and their modifications, and variations may be created by appropriately combining a plurality of components disclosed in each of the embodiments and modifications. For example, some components may be excluded from among all components indicated in each embodiment and modification, or components indicated in different embodiments and modifications may be appropriately combined.

According to the present disclosure, it is possible to capture features peculiar to medical image data.

What is claimed is:

1. An image processing apparatus comprising:
a memory; and
a processor comprising hardware, the processor being configured to output a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group,
wherein the similar image group is different from the image group to be classified in the main learning.

2. The image processing apparatus according to claim 1, wherein the shape of the object is a tubular structure in a living body.

3. The image processing apparatus according to claim 2, wherein
the target image group is an image group obtained by capturing a lumen in the living body in a predetermined section, and
the similar image group is an image group obtained by capturing the lumen in the living body in a section different from the section of the target image group.

4. The image processing apparatus according to claim 2, wherein the similar image group is a mimic organ image group obtained by capturing a mimic organ that mimics the tubular structure.

5. The image processing apparatus according to claim 1, wherein
the tissue structure of the object is a mucosal structure of an organ system, and
the similar image group is an image group obtained by capturing a mucosal structure of an organ system identical to the target image group.

6. The image processing apparatus according to claim 5, wherein the organ system is any one of digestive, respiratory, urinary, and circulatory organs.

7. The image processing apparatus according to claim 1, wherein the imaging system of the device is an imaging system of an endoscope.

8. The image processing apparatus according to claim 7, wherein the similar image group is an image group obtained by capturing a mimic organ that mimics a predetermined organ by the imaging system of the endoscope identical to the target image group.

9. The image processing apparatus according to claim 1, wherein the preliminary learning is performed based on a result of basic learning and the similar image group, the basic learning being performed based on a dissimilar image group different from the target image group in characteristics.

10. A learning device comprising:
a processor comprising hardware, the processor being configured to:
perform preliminary learning based on a similar image group similar in at least one of characteristics of a shape of an object in a target image group to be learned, a tissue structure of an object in the target image group, and an imaging system of a device that captures the target image group; and
perform main learning based on a preliminary learning result by the preliminary learning unit and the target image group,
wherein the similar image group is different from the image group to be classified in the main learning.

11. An image processing method executed by an image processing apparatus, the method comprising
outputting a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group,
wherein the similar image group is different from the image group to be classified in the main learning.

12. A method of creating a classification criterion executed by a learning device, the method comprising
outputting, as the classification criterion, a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group,
wherein the similar image group is different from the image group to be classified in the main learning.

13. A learning method executed by a learning device, the method comprising:
performing preliminary learning based on a similar image group, acquired from a recording unit, similar in at least one of characteristics of a shape of an object in a target image group to be learned, a tissue structure of an object in the target image group, and an imaging system of a device that captures the target image group; and
performing main learning based on the target image group acquired from the recording unit and a result of the preliminary learning,
wherein the similar image group is different from the image group to be classified in the main learning.

14. A non-transitory computer readable recording medium on which an executable program is recorded, the program instructing a processor of an image processing apparatus to execute
outputting a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group,
wherein the similar image group is different from the image group to be classified in the main learning.

15. A non-transitory computer readable recording medium on which an executable program is recorded, the program instructing a processor of a learning device to execute
outputting, as a classification criterion, a result of classifying an image group to be classified based on a result of main learning performed based on a result of preliminary learning and a target image group to be learned, the preliminary learning being performed based on a similar image group similar in at least one of characteristics of a shape of an object in the target image group, a tissue structure of an object in the target image group, and an imaging system of a device that captures the target image group,
wherein the similar image group is different from the image group to be classified in the main learning.

16. A non-transitory computer readable recording medium on which an executable program is recorded, the program instructing a processor of a learning device to execute:
performing preliminary learning based on a similar image group, acquired from a recording unit, similar in at least one of characteristics of a shape of an object in a target image group to be learned, a tissue structure of an object in the target image group, and an imaging system of a device that captures the target image group; and
performing main learning based on the target image group acquired from the recording unit and a result of the preliminary learning,
wherein the similar image group is different from the image group to be classified in the main learning.
